SEQUENCE LISTING (8 sequences)

SEQ ID NO: 1 - protein, 91 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQM

SEQ ID NO: 2 - protein, 47 aa, Artificial Sequence
  Synthetic peptide
CSLAVVLQRRDWENPGVTQLNRLAAHPPFASWRNSEEARTDCPSQQL

SEQ ID NO: 3 - protein, 981 aa, Artificial Sequence
  Synthetic peptide
RTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 4 - protein, 1159 aa, Artificial Sequence
  Synthetic peptide
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 5 - protein, 2243 aa, Artificial Sequence [sequence omitted]

SEQ ID NO: 6 - DNA, 5424 bp, Artificial Sequence
  Synthetic oligonucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccgcggccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 7 - protein, 6 aa, Artificial Sequence
  Synthetic peptide
GNGGNA

SEQ ID NO: 8 - DNA, 12156 bp, Artificial Sequence
  Synthetic oligonucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgggaaacgggggaaacgcaatgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagtggaaacgggggaaacgcacgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataagcggccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

(19) United States
(12) Patent Application Publication
(10) Pub. No.: US 2012/0202222 A1
(43) Pub. Date: Aug. 9, 2012

(54) ENZYME FRAGMENT COMPLEMENTATION ASSAYS FOR MONITORING THE ACTIVATION OF THE VOLTAGE-GATED POTASSIUM ION CHANNEL HERG

(75) Inventors: Jeffrey Kenneth Horton, Cardiff (GB); Peter James Tatnell, Cardiff (GB)

(73) Assignee: GE HEALTHCARE UK LIMITED, LITTLE CHALFONT (GB)

(21) Appl. No.: 13/501,782

(22) PCT Filed: Oct. 12, 2010
(86) PCT No.: PCT/EP2010/065260

§ 371 Date: Apr. 13, 2012

(30) Foreign Application Priority Data
Oct. 13, 2009 (GB) 0917877.3

(51) Int. Cl. G01N 33/566; C12N 15/63; C12N 15/62; C12N 5/10; C07K 19/00

(52) U.S. Cl. 435/7.21; 435/7.9; 435/325; 530/350; 536/23.4; 435/320.1

(57) ABSTRACT

The present invention provides methods and cell based assays for testing for the binding of a ligand to a human Ether-a-go-go-related (hERG) voltage-gated potassium ion channel protein in an enzyme complementation assay. The invention is of particular use in toxicological and drug screening, particularly for high throughput screening.

### FIELD OF THE INVENTION

The present invention relates to the field of cell biology, molecular biology and toxicology. In particular, the invention relates to cardio-toxicology and to methods to assess the activity of the voltage-gated potassium ion channel hERG (also known as KCNH2 or Kv11.1).

### BACKGROUND TO THE INVENTION

### Ion Channels

Ion channels are fundamental to normal physiological processes allowing the passage of charged ions through hydrophobic membranes with exquisite specificity, at speeds close to that of diffusion. They are present in the membranes of almost all living cells from simple bacteria to highly specialized cell types such as neurons, muscle cells etc. They are essential for important physiological processes such as sensory transduction, action-potential generation, muscle contraction etc. When they malfunction, for example through mutation or disease, there can be serious consequences which is often life threatening.

Whenever charged molecules such as inorganic ions etc., are transported across membranes, their movement constitutes an electrical current that generates a voltage difference across the membrane. All living cells have the ability to exploit a trans-membrane potential as an intermediate in the storage of energy and the synthesis of ATP. However, specialized cells such as neurons and muscle cells (known collectively as excitable cells) have the additional ability of generating fast electrical signals based upon these voltage differences. These fast electrical signals are made possible by the homeostatic mechanisms that establishes the standard environment of animal cells i.e. high sodium ion (Na⁺) concentration in the blood and extra-cellular fluid and high potassium ion (K⁺) and calcium ion (Ca²⁺) (but low Na⁺) concentrations in the cytoplasm. Concentration gradients (separated by the cell membrane) are maintained by active transporters which prepares the way for rapid changes in membrane voltage generated by passive transport mechanisms through ion channels. These pore forming channel allow ions to flow down an electrochemical gradient. For example an open Na⁺ channel permits Na⁺ to flow down a concentration gradient into a cell, making the intracellular voltage more positive. Opening a K⁺ channel permits K⁺ to flow out of the cell restoring the electrochemical voltage balance. The relatively small size of a cell allows the voltage to be changed extremely rapidly with only minor changes in ion concentration.

The ultimate specialization of electrical signaling is the action potential. This is a millisecond-long electrical signal that is capable of propagating at speeds of meters per second along a nerve fiber.

### Voltage-Gated K⁺ Channels

See Yellen, G. (2002), Nature, 419, 35-42 for details.

Potassium selective channels have diverse structures and functions including maintaining the resting membrane potential in all cell types and the termination of the action potential in excitable cells. The voltage-gated K⁺ channels at their simplest are homo-tetrameric channels. Each of the subunits consists of a voltage-sensor and contributes residues to the central pore. The standard voltage-gated K⁺ channel contains six trans-membrane regions, with both amino and carboxyl termini on the intracellular side of the membrane. These signaling proteins perform several functions. Fast and selective ion permeation is regulated by the opening and closing of the pore which are essentially a set of conformation changes called gating. Gating is coupled to a sensing mechanism which detects trans-membrane voltage but can also be geared to cyclic nucleotides for certain voltage-gated K⁺ channels.

Voltage-gated K⁺ channels allow ions to pass at speeds that are similar to those exhibited by aqueous diffusion rates. Physical properties such as ion selectivity and speed are crucial for correct biological function. The specificity of a channel determines current flow e.g. Na⁺ and Ca²⁺ ions flow inward and thus carry positive charge into the cell. Most voltage-activated channels are sensitive to positive electrochemical charge. An influx in to the cell of positive ions activates voltage-gated K⁺ channels and these initiate charge restoration or regenerative excitation i.e. K⁺ ions flow outwards (or Cl⁻ ions flow inwards) thereby reducing the internal positive charge, terminating the cell excitation process. In a mammalian neuron a typical action potential requires the flow of millions of ions per millisecond. To accomplish this rate requires the ion channel to exhibits a high ion conductance rate, whilst maintaining high ion selectivity.

Voltage-gated K⁺ channels exhibit four architectural features that maintain ion stability during translocation. First, the channels requires water and rather than maintaining a narrow pore for the entire thickness of the cell membrane part of the ion permeation pathway is broad and contains a large water filled cavity. Second, the channel stabilizes the ions and achieves selectivity by using electrostatic influences such as helix dipoles in which one end of the helix is more negative relative to the other. The intracellular side of voltage-gated K⁺ channels is arranged so that the more negative end of the four α-helix dipoles (contributed by each sub-unit) are positioned towards its centre. These produce a preferential stabilization of cations near the entrance of the narrow internal selectivity filter. This arrangement is mirrored by Cl⁻ channels however these adopt a structure in which the positive-charged ends of multiple helices are arranged pointing towards their central site. A third feature of the voltage-gated K⁺ channel that facilitates ion selectivity and permeation is the creation of a series of customized polar oxygen cages. As a K⁺ ion diffuse through water it is surrounded by a cage of polar oxygen atoms derived from the surrounding water molecules. In the selectivity filter each K⁺ ion is surrounded by two groups of four oxygen atoms in a similar arrangement to that exhibited when the ion is in a solution of water. These oxygen atoms are derived from the carbonyl oxygen atoms of the peptide residues that constitute the selectivity filter loops. Each of the four subunits contributes residues that generate the selectivity filter. Finally K⁺ ions pass through the channel in single file with simultaneous occupancy by multiple ions. The mutual electrostatic repulsion between adjacent ions destabilizes ion stability in the pore permitting a favorable interaction between the channel and the ion that facilitates ion selectivity and translocation without producing tight binding that would impair rapid ion permeation. The structural features of voltage-gated K⁺ channels are therefore perfectly adapted to fit their function. These features overcome the electrostatic problem of stabilizing ions without making them more stable than they are in water, by using a large cavity filled with water and helix diploes to counteract the unfavorable environment present within lipid membranes. The preferential selection and stabilization of K⁺ in preference to Na⁺ is achieved by precisely matching the arrangement of oxygen atoms around a solvated K⁺ ion.

The Voltage-Gated K⁺ Channel—hERG
(See http://www.hergchannel.com for General Information About hERG Channel)

Much of the current understanding on the structure and function of the human ether-a-go-go-related (hERG also known as KCNH2 or Kv11.1) voltage-gated K⁺ channel originates from investigations on other voltage-gated K⁺ channels. These studies have included biophysical investigations on the Drosophilia shaker protein, X-ray crystal structures of i) several bacterial K⁺ channels and ii) the mammalian Kv1.2 K⁺ channel and structure function analysis of mutant hERG channels. All have provided valuable insights into the structural basis of selective K⁺ permeability and channel gating. See Sanguinetti, M. C., et al., 2006, Nature, 440, 463-469).

The hERG voltage-gated K⁺ ion channel is known for its contribution to the electrical activity of the heart by coordinating cardiac beating (i.e. hERG mediates the repolarizing current in the cardiac action potential). In the human it is expressed in multiple tissues and cell types, including neural, smooth muscle and several tumor cells. It is highly expressed in the heart and this is where its function is best characterized and understood. Two other ERG channels (ERG2 and 3) are expressed in the mammalian central nervous system and in these neural derived tissues hetero-tetrameric hERG channels can form composed of a combination of any of the three hERG subunits.

The hERG genomic structure consists of 15 exons, spanning about 19 kb of genomic DNA. The translated protein is 1159 amino acids in length. The hERG channels are believed to form homo-tetramers of identical six α-helical trans-membrane spanning domains, with a cluster of positive charges localized in the S4 domain acting as the putative voltage sensor (Thomas, D., et al., 2006, Curr. Pharma. Design, 12, 2271-2283).

Inherited mutations in hERG affect cardiac electrical activity causing a condition known as long QT syndrome (LQTS). This is a cardiac re-polarization disorder that predisposes affected individuals to arrhythmia (rapid irregular heart beats) that can be lethal. It is defined by prolongation of the QT interval as measured by an electrocardiogram (ECG). The QT interval is the time required for ventricular re-polarization during a single cardiac cycle, delayed re-polarization increases the risk of a condition known as “torsade de pontes”. This is a cardiac arrhythmia characterized by an ECG recording as a continuous sine wave-like. LQTS affects an estimated 1 in 5,000-10,000 individuals worldwide and is caused by a mutation in the hERG subunit. This and similar mutations reduce the outward K⁺ conductance, thereby slowing the re-polarization rate of the action potential resulting in the electrical instability that can generate torsade de pontes. Approximately ˜300 LQTS-associated mutations in hERG have been described (see http://pc4.fsm.it:81/cardmoc/ for details). The functional consequence of the majority of hERG mutations is either the miss-folding of the subunits or the incorrect trafficking of the channel to the cell membrane. Common mutations can also affect hERG gating and in some in-vitro instances a dominant-negative mutant subunit is generated that can co-assembles with an active wild-type sub-unit suppressing functional channel activity.

One naturally-occurring hERG mutation (Asn588Lys) represents a gain of function that actually abolishes channel inactivation. It hastens cardiac re-polarization by shortening the QT interval and can cause ventricular fibrillation and possible death. The fact that both loss- and gain-of-function mutations in hERG can cause lethal arrhythmia emphasizes that the normal electrical activity of the heart requires a balanced expression of a range of different ion channels including hERG.

A similar drug-induced arrhythmia as exhibited by naturally-occurring LQTS can be induced by the administration of some hERG-channel blockers for example quinidine and dofetilide. Other non-cardiac medications such as certain antihistamines and antibiotics can also trigger ventricular arrhythmia and death by blocking the hERG channel. These drug-induced disorders have in the past resulted in the withdrawal of the therapeutic agents.

### Cardiac Action Potential

The action potential of ventricular myocytes can be divided into five distinct phases. Phase 0, represents the activation of inward Na⁺ channels, triggering a rapid depolarization (approximately −90 to +50 mV) of the membrane. Phase 1, represents a rapid re-polarization (to ˜+10 mV) but this only lasts for a few milliseconds. This is followed by the much slower phase 2 or plateau phase of re-polarization. This plateau phase is prolonged because the K⁺ channels are slow to activate and/or have a reduced conductance at positive trans-membrane potentials. A prolonged re-polarization phase ensures the entry of extra-cellular Ca²⁺ into the myocyte for optimum excitation-contraction coupling. It also makes the cardiac muscle refractory to premature excitation which is an important control against the generation of arrhythmia. Phase 3 terminates the action potential and returns the membrane potential to its Phase 4 resting level of ˜−90 mV. The important component of the phase 3 re-polarization stage of the action potential is the rectifier voltage-gated K⁺ current conducted by hERG (Sanguinetti, M. C., et al., 2006, Nature 440, 463-469).

FIG. 1 shows a human ventricular action potential. An inward Na⁺ current triggers a rapid depolarization of the membrane (phase 0). Repolarization proceeds rapidly at first (phase 1), followed by a slower rate of repolarization (phase 2). The third phase ends the action potential and returns the membrane potential to the resting level (phase 4).

At negative membrane potential the hERG channels are in a non-conducting or closed state. Depolarization of the membrane to a less negative (or more positive) value induces the channel to open and allows the outward diffusion of K⁺ in accordance with its electrochemical gradient. As the membrane potential progressively depolarizes to a positive state hERG adopts the non-conducting configuration known as the inactivated state. The rapid membrane depolarization results in the rapid inactivation of hERG (phase I as described above). Inactivation occurs significantly faster than its activation and thereby reduces the overall outward K⁺ conductance at depolarized or positive potentials. As the membrane re-polarizes (and becomes more negative), a slow gradual activation of the hERG channel occurs. The effect of this is to essentially prolong phase 2 of the action potential. The full recovery of hERG channel activity initiates phase 3 re-polarization of the action potential.

hERG—Structure Function Relationship

See Perrin, M. J. et al., 2008, Prog. Biophy. & Mol. Biol. 98, 137-148 for details. The crystal structures of the bacterial K⁺ channels KcsA, MthK and KvAP (Doyle, D. A., et al., 1998, Science, 280, 69-77, Jiang, Y, et al., 2002, Nature, 417, 523-526 and Jiang, Y., et al., 2002, Nature, 423, 33-41 respectively) in combination with that of the mammalian Kv1.2 voltage-gated K⁺ channel (Long, S. B., et al., 2005, Science, 309, 897-903) have increased the understanding of the structural basis of voltage-gated K⁺ channel function. The K⁺ channels are formed by the co-assembly of four identical α-subunits each containing six α-helical trans-membrane domains (S1-S6) that form two functionally distinct domains, one that senses trans-membrane potential (S1-S4) and one that forms the K⁺ selective pore (S5-S6 including the pore domain).

FIG. 2 shows the structure of a single hERG subunit containing six α-helical trans-membrane domains (S1-S6). The S4 domain contains multiple basic amino acids while the S1-53 domains contain many acidic residues. Amino acids from each domain form electrostatic salt bridges during ion channel gating. The per-arnt-sim (PAS, residues 1-137), voltage sensor (residues 407-545), pore domain (residues 545-665), cyclic nucleotide binding domain (cNBD residues 742-844) are highlighted.

The extra-cellular pore domain forms the K⁺ selectivity filter and this is design for the specific selection and conduction of K⁺. In voltage-gated K⁺ channel the selectivity filter is defined by a conserved sequence Thr-Val-Gly-Tyr-Gly located at the carboxy-terminal end of the pore helix. In each subunit, the side-chain hydroxyl group of the Thr residue and the carbonyl oxygen atoms of the other four residues face towards the opening of the ion-conduction channel. Together these oxygen atoms form several octahedral binding sites that coordinate K⁺ ions arranged in a single file and separated by a single water molecule. In hERG, the Thr and Tyr residues are substituted with Ser and Phe.

Below the selectivity filter, the pore widens into a large water-filled region, called the central cavity. This is lined by the S6 α-helices. In the closed state, the four S6 domains essentially fill the space near the cytoplasmic interface forming a narrow aperture that is too small to permit entry of K⁺ from the cytoplasm. In response to the membrane depolarization, the S6 α-helices splay outwards thus increasing the diameter size of the aperture and thereby facilitating K⁺ movement to the extra-cellular compartment.

Sequence analysis and hydropathy plots of the voltage-gated K⁺ channel hERG indicate that, in common with other voltage-gated K⁺ channels hERG is a homo-tetramer with each subunit containing six trans-membrane domains (S1-S6). Recent high resolution crystal structures of other K⁺ channels have shown that the S5, the pore helix and S6 domains contribute to form the pore domain which assemblies around a selective and high throughput ion conduction pathway. The S1-S4 trans-membrane domains from each subunit form the voltage sensing domains which moves within the membrane in response to trans-membrane voltage and thereby regulate the opening and closing of the pore domain.

Unique to the hERG family is an extra-cellular domain of ˜40 amino acids between S5 and the pore or P-domain (the S5P linker) which has been shown to have an amphipathic helical arrangement. In addition, to these trans-membrane regions, hERG also possesses intracellular N and C-terminal domains. The N-terminal contains a Per-Arnt-Sim (PAS) domain. These domains are common in plants and bacteria where they form signal sensor domains. In hERG the PAS domain is believed to be involved in the deactivation of the ion channel. The C-terminal tail contains a cyclic nucleotide binding domain (cNBD). The function of this domain in the context of hERG activity is not fully understood, as cAMP binding to this domain has little effect on channel activity.

Gating of hERG K⁺ Channels

Voltage-gated K⁺ channels can exist in at least three different configurations closed, open and inactivated. Transition between closed and open configurations corresponds to constriction and widening of an activation gate in the intracellular portion of the S6 helix. C-type inactivation that occurs in hERG results from structural changes in the selectivity filter positioned between the pore helix and S6 which causes constriction of the conduction pathway and interruption of K⁺ ion translocation.

Despite significant sequence homology to other voltage-gated K⁺ channels, hERG channels have very distinct kinetics, characterised by slow activation but very rapid and voltage-dependent inactivation. As a result of slow activation and simultaneous fast inactivation at depolarised potentials little outward K⁺ current flows through hERG during repolarisation of the AP (Phases 1 and 2). Reduced outward current at these potentials contributes to maintenance of the plateau of the action potential allowing sufficient time for Ca²⁺ entry. This renders the cell refractory to premature excitation. As the membrane repolarises during phase 3, hERG channels recover from inactivation much faster than they deactivate thereby passing more current, with the outward current peaking at ˜−40 mV. This outward current through hERG is the most important determinant of the termination of the plateau phase of the action potential. The current then decreases as a result of a combination of a decrease in the driving force for K⁺ and slow deactivation. In the context of a premature stimulus this slow rate of deactivation is crucial. The onset of the premature stimulus causes a large increase in outward K⁺ current as a result of the much larger electrochemical driving force. Due to fast inactivation at depolarised potentials this large outward current will be very transient. The large outward current in response to a premature stimulus would oppose cellular depolarisation and thereby help to suppress the propagation of premature beating and arrhythmias.

The activation gate in voltage-gated K⁺ channels is formed by regions of the S6 trans-membrane helices close to the intracellular membrane. Crystal structures indicate that in the closed state these helices form a bundle, such that the opening is too narrow to permit passage of K⁺ ions. Transition to the open state occurs when these helices splay outwards via a kinking mechanism at a gating hinge. This causes pore dilation facilitating the passage of K⁺ ions. In the voltage-gated K⁺ channels Kv1-Kv4, this hinge is formed by a conserved ProValPro motif. hERG however, lacks this motif but possesses a ProValGly. It has been shown that the mutation Gly657Pro results in a permanently open channel, presumably by locking the helices in the kinked or open position.

In voltage-gated ion channels, transitions between the open and closed state are regulated by the S1-54 voltage sensitive domain. Within this domain the primary voltage sensor is thought to be the ‘paddle motif’ comprising the extra-cellular half of S3 and S4. In the hERG S4 domain, six basic amino acids are positioned every 3 residues between positions 525 and 538, these provide the positive charge required to sense changes in membrane voltage. Of these residues, Lys525, Arg528 and Lys538 are the most important contributors to voltage sensing for slow activation. In addition to these positive charges, acidic residues in S1-S3 form salt bridges with the basic residues in S4 to stabilize the voltage sensitive domain in the open (Asp460 and Asp509) or closed (Asp411) conformation.

Voltage sensing involves movement of the voltage sensitive domain between an up/open and a down/closed state, mediated by electrostatic forces exerted on S4 positive charges due to changes in the trans-membrane electric field. However, the exact structural rearrangements that occur between the ‘up’ and ‘down’ states and more specifically the magnitude of movement of the voltage sensor are not fully understood. The structure of the mammalian Kv1.2 channel demonstrates the general organization of the voltage sensitive domain relative to the “open” pore domain, i.e. with the voltage sensor in the ‘up’ conformation. However, without a K⁺ channel crystal structure corresponding to the closed channel or voltage sensor in the ‘down’ state it is very difficult to understand the nature of this structural rearrangement.

The most recent models involve a ˜180° rotation of the S4 as they move outward by 7 Å, followed by a tilting of the S4 and S5 helices. What is generally accepted is that the structural rearrangements which occur in the voltage sensitive domain are transferred to the pore domain via a physical interaction of the S4-S5 linkers with the cytoplasmic terminals of the S6 helices, which acts to open and close the activation gate.

Sequence alignments and hydropathy plots for hERG suggest that the overall structure of the voltage sensitive domain is homologous to that of other voltage-gated K⁺ channels. Experimental evidence suggests that the S4 helix in hERG is loosely packed and most likely lipid exposed, as it is in the crystal structure of Kv1.2. However, the hERG kinetics of activation is very different. Gating currents measured from hERG, corresponding to movement of the voltage sensor charges across the membrane, showed a slow time course corresponding with the slow activation time. Confirmatory evidence for slow activation underlying slow movement originates from the slow movement of a fluorophore attached to the extra-cellular end of the S4 domain after depolarisation.

Structural Basis of hERG-Channel Gating

See Perin, M. J. et al., 2008 Prog. Biophy. & Mol. Biol. 98, 137-148 and Sanguinetti, M. C. et al., 2006, Nature, 440, 463-469 for details. Three established mechanisms exist by which the voltage-gated K⁺ channels can close: two of them involve a conformational constriction of the permeation pathway or channel and one involves conditional plugging of the pore by an auto-inhibitory part of the channel protein.

First the channel can close by pinching shut at the intracellular entrance. This intracellular or S6 gate obstructs the entrance (from the cytoplasmic side) to the water-filled cavity in the centre of the K⁺ channel pore. The extra-cellular ends of the four S6 helices form a cradle for the selectivity filter whereas the intracellular ends converge to form a bundle positioned below the cavity. The nature of the S6 opening motion has been made apparent by observing the crystal structure of the bacterial MthK K⁺ channel (Jiang, Y. et al., 2002, Nature, 417, 523-526). In this structure the channel is in the open configuration and the homologues to the hERG S6 helices are splayed wide open with no apparent constriction or blocking between the intracellular solution and the selectivity filter. Therefore an explanation for the S6 gating motion is that the S6 helices swing open from an apparent hinge located at a highly conserved glycine residue. It is believed that general voltage-gated K⁺ channels closing utilizes the highly conserved Pro-X-Pro sequences that provides a fixed bend allowing the S6 gate to connect with the trans-membrane voltage sensor. An important consequence of the S6 gating mechanism is its interaction with channel blockers. These act by binding to sites within the cavity. Due to the S6 gate, these blockers can enter the channel only after the channel has been opened by a voltage. Therefore many blockers are trapped in the cavity when the S6 gate closes.

A second mechanism for pore closing uses the S6 gate to regulate the binding of an auto inhibitory peptide that is part of the K⁺ channel. The N-type inactivation (or ball and chain mechanism) occurs in several known voltage-gated K⁺ channels and can involve either the N-terminus of the α-subunit or the N-terminal region of an associated subunit. This inhibitory action can be disrupted by proteolysis or the removal of the N-terminal sequences by recombinant DNA technology. Inhibition can be restored by the addition of an intracellular expressed peptide of the same sequence. The peptide appears to inhibit the channel by a simple physical blocking mechanism.

A third mechanism for closing the pore is to pinch shut at the selectivity filter itself i.e. a selectivity filter/pore gate (or C-type inactivation). The structural details of C-type inactivation probably involve the carbonyl oxygens of the selectivity filter. On pore closing these moieties project obliquely causing a partial collapse of the filter. In the open configuration these point towards the central axis. The basic functioning of the S6 gate and the selectivity filter gate appear to be conserved among the different voltage-gated K⁺ channels. The S6 gate moves in response to a trans-membrane voltage sensor (domains S1-S4) and the selectivity filter reacts to the change in the S6 by facilitating channel opening or closing.

As the channel switches between closed and open configuration i.e. during voltage gating as many as four K⁺ are transferred across the membrane per subunit. The only sequence motif to be conserved across all voltage-gated ion channels (Na⁺, K⁺ and Ca²⁺) is the fourth trans-membrane region or S4 domain in which every third residue has a positively charged Arg or Lys. This arrangement of positive charges in the trans-membrane region are energetically unfavorable and therefore to compensate negative countercharges are present in the S2 trans-membrane region and the water filled canal positioned at the end of each S4 trans-membrane region. Therefore during changes in the membrane electrochemical voltage and the unique S4 domain arrangement a position accessible from the intracellular site at negative voltage can become accessible from the outside at positive voltage. Little of the S4 domain actually appears too buried in the membrane for example when one charged residue is positioned at the extra-cellular side, a site only six residues away in the primary amino acid sequence is accessible from the intracellular environment. This readily-accessible S4 domain arrangement is consistent with the presence of a water-filled cavity accessible from both ends of the pore.

Altogether the α-helical domains S1-54 are presumed to form the voltage sensor domain. The pattern of the charged residues at every third position suggested a possible helical screw motion. The S4 domain therefore would advance screw-like moving each solvated K⁺ to the position of the next charge thereby maintaining the charge distribution in the channel core while producing an overall translocation of K⁺ across the membrane.

Cloned and expressed recombinant hERG channels have provided details on the structural basis of its biophysical properties. The trans-membrane electrical field provides the force that drives the gating of K⁺ voltage channels. The S4 α-helical domain of hERG represents the voltage-sensing domain. When the membrane is depolarized, the S4 moves outward facilitating channel opening. Voltage-dependent movement of the hERG S4 domain can be detected as a small change in the fluorescence of an attached fluorophore (Smith, P. L. et al., 2002, J. Gen. Physiol. 119, 275-293). This study revealed two components of the S4 domain movement. Firstly, a slow movement that accounts for the slow rate of hERG channel activation. This implies that a large energy barrier must be overcome to facilitate the transitions between the multiple closed and the channel open states. A mutagenesis program of the S4 domain identified Arg531 which is the fourth residue in the S4 domain as the most important residue for voltage sensing and thus channel opening (Subbiah, R. N., 2005, J. Physiol., 569, 367-379).

Negatively charged residues in S1-53 form transient salt bridges with the basic residues in the S4 domain and thereby stabilize the closed, intermediate and open states of the hERG channel (Silverman W. R., 2003, P.N.A.S. 100, 2935-2940). The outermost acidic Asp residues in S2 and S3 form a coordination site for extra-cellular divalent cations. This arrangement shields against salt-bridge formation thus stabilizing the closed conformation, shifting the voltage dependence of hERG opening to less negative potentials.

The crystal structure of Kv1.2 (Long, S. B. et al., 2005, Science, 309, 897-903) reveals that the S4 voltage-sensing domain is linked to the pore module by the S4-S5 linker. This is an amphipathic α-helix that runs parallel to the membrane near the cytoplasmic interface near the C-terminal portion of the S6 α-helix within the same subunit. It is proposed that the S4-S5 linker functions as a lever, (driven by voltage induced changes in S4), which pushes against the S6 helices to regulate channel opening. Studies in a variety of voltage-gated K⁺ ion channels, including hERG, support an important role for the S4-S5 linker in channel gating. In hERG, an electrostatic interaction between specific residues in the S4-S5 linker (Asp540) and the C-terminal region of the S6 domain (Arg665) stabilizes the closed channel conformation.

Inactivation Gating of hERG

Inactivation in voltage-gated K⁺ channels may occur by N-type (ball and chain) or C-type (pore collapse). The available evidence suggests that inactivation of hERG is via the C-type mechanism. Inactivation is sensitive to external K⁺ concentration. These ions acts to physically prevent ‘collapse’ of the selectivity filter. However, hERG inactivation is significantly faster than the C-type inactivation observed in other voltage-gated K+-channels, and is intrinsically voltage-dependent. Several studies have focused on identifying the molecular basis of the voltage-sensitivity of hERG inactivation and determining the relationship between activation and inactivation gating.

Other domains may contribute to the voltage-sensitivity of inactivation. Two serine residues, Ser620 and Ser631 in the P-domain are critical for inactivation. While the Ser620Thr mutant abolishes inactivation completely and Ser631Ala causes a 100 mV depolarising shift in the voltage-dependence of inactivation, neither of these mutants affects activation. Conversely, charge manipulation in the S5P domain can readily alter the voltage-sensitivity of inactivation in hERG, while having minimal effect on activation. Therefore the voltage sensor for inactivation is probably different from that for activation. Perrin, M. J. et al., 2008, Prog. Biophys. & Mol. Biol. 98, 137-148 have suggested that the amphipathic α-helix in the S5P domain of hERG is involved in the voltage sensing process associated with inactivation and that inactivation involves the relative movement of the S5P amphipathic helix and the P-domain. This is supported by Jiang, M. et al., 2005, J. Physiol. 569, 75-89, who demonstrated that the S5P domain in hERG possesses a highly dynamic structure that could easily adopt different conformations between the open and inactive states.

Mechanisms of hERG Channel Dysfunction

Any mutations that affect hERG channel function could have a deleterious effect on cardiac electrical activity. To date nearly 300 hERG channel mutations have been identified (see http://www.fsm.it/cardmoc/;).

The magnitude of the current associated with hERG activation is determined by i) the total number of channels, ii) the percentage of channels in the open configuration and iii) the ion conductance of the channels. Any mutations may result in the loss of function by the following; defective or reduced synthesis, defective trafficking, defective gating or defective ion conductance.

Defective synthesis—Approximately 25% of all hERG mutations result in premature termination codons. A second mechanism involves microRNA-mediated mRNA silencing. Xiao, J., et al., 2007, J. Biol. Chem. 282, 12363-12367, have shown that up-regulation of the microRNA-133 that is associated with diabetic hearts is responsible for a significant decrease in hERG mRNA expression.

Defective trafficking—hERG is expressed as a single polypeptide that becomes core-glycosylated and assembled into the tetramer in the endoplasmic reticulum. The hERG tetramerisation motif is located in the C-terminal domain. In the Golgi complex further glycosylation occurs. Trafficking from the ER is dependent on the shielding of the ER retention signal (Arg-X-Arg) and the functional presentation of the ER exit signal (Asp/Glu-X-Asp/Glu) to facilitate hERG incorporation into transport vesicles. Mutations that affect either subunit assembly in the ER or trafficking from the ER to the plasma membrane will all result in trafficking failure. The majority of LQTS 2 mutants (−80%) are due to trafficking defects (Anderson, et al., 2006, Circulation, 113, 365-373).

Defective gating—Altered gating characteristics can lead to reduced hERG current by either reduced activation or enhance inactivation For example the Arg534Cys mutation when expressed in Xenopus results in an increase in the open channel configuration.

Defective ion conductance—Mutations in the vicinity of the selectivity filter of hERG result in either an altered ionic selectivity or reduced ion conductance.

Any defect in hERG channel activity has the potential cause arrhythmic. The reduction level required causing a significant increase in the risk of arrhythmia and sudden death is at present unknown. The International Registry of LQTS has performed a number of studies directed at assessing this type of risk e.g. the longer the QT interval and the earlier the age of onset of symptoms the greater the risk of sudden death (Priori, S. G. et al., 2003, N. Engl. J. Med. 348, 1866-1874. In addition, the Registry is conducting in-vitro mutation assays aimed at assessing the reduction in hERG activity associated with any given mutation. A single point mutation causing defective synthesis would be expected to result in a 50% reduction of hERG activity as the defective protein is expressed in the presence of a wild-type hERG channel. However a dominant negative trafficking mutant may be expected to present a more severe phenotype.

Drug-Induced Block of hERG Channels

Drug-induced prolongation of the QT-interval may occur in response to a compound designed to block cardiac repolarizing currents e.g. dofetilide etc. Unfortunately, QT prolongation may also arise as an unwanted side effect of a compound designed to act at non-cardiac sites and this is the most common cause of withdrawal or restriction in already-marketed drugs. Clinically-relevant drug-induced QT prolongation generally involves either the blockage of hERG or interruption to its trafficking.

Certain medications can generate physiological effects similar to those exhibited by inherited LQTS i.e. prolonged QT and torsade de pontes. Induction of torsade de pontes by drugs other than anti-arrhythmic agents is a rare event for example cisapride-induced torsade de pontes occurs in about 1 out of 120,000 patients (Vitola, J. et al., 1998, J. Cardiovasc. Electrophysiol. 9, 1109-1113). Cisapride is normally used to treat non-life threatening disorders of the gastro intestinal tract. The occurrence of similar drug-induced prolonged QT syndromes and torsade de pontes prompted Pharmaceutical Regulatory Agencies to remove from the market or relegate to “restricted use” several drugs, including cisapride, sertindole, grepafloxacin, terfenadine and astemizole.

Inherited LQTS and torsade de pontes can be caused by the loss-of-function of several cardiac K⁺ channels. However, drug-induced QT prolongation and torsade de pontes are caused by either the direct blockage of hERG channels, interference with hERG-channel trafficking or drug-drug interactions that ultimately lead to a reduction in hERG-channel current activity (see http://www.qtdrugs.org for details of pro-arrhythmic drugs).

Drug-induced blockage of hERG channels occurs with a range of chemicals possessing diverse structures that encompass several therapeutic drug classes, including anti-arrhythmic, psychiatric, antimicrobial, antihistamine etc. Indeed, the hERG channel appears to be unusually susceptible to drug-induced blockage compared with other K⁺ voltage channels. Pharmaceutical companies are required to routinely screen compounds for hERG-channel activity early during preclinical safety assessment.

An Ala-scanning mutagenesis approach was used to identify hERG residues that interact with such drugs. Residues within the pore module were individually mutated to Ala, and the resulting mutant channels assayed for sensitivity to potent hERG blockers (Mitcheson, J. S. et al., 2000, P.N.A.S. 97, 12329-12333). Ala mutation of the two polar residues (Thr623 and Ser624) located at the base of the pore helix and two aromatic residues (Tyr652 and Phe656) located in the S6 domain of the hERG subunit to Ala residues significantly decreased the affinity of the anti-arrhythmic drug MK-499. The same residues were subsequently discovered to be important for binding of cisapride, terfenadine and several other drugs from diverse chemical and therapeutic classes. The side chains of each of these residues are orientated towards the large central cavity of the channel which is consistent with the observation that hERG channels are blocked by these drugs only after channel opening.

The two pore helix residues (Thr623 and Ser624) are highly conserved in most K⁺ voltage-gated channels and thus cannot easily explain the promiscuous blocking by drugs of hERG. However, the two S6 residues (Tyr652 and Phe656) are not conserved and most K⁺ voltage-gated channels have an Ile and a Val in homologous positions. Therefore the highly promiscuous and high-affinity binding of blocking drugs to the hERG channel is probably related to the presence of these specific S6 residues.

Potent blockage of hERG by cisapride and terfenadine requires an aromatic residue in position 652, suggesting the possible electrostatic interaction between a positively charged N of the drug and the π-electrons of Tyr 652. A hydrophobic attraction between Phe656 and cisapride was also identified as being important for channel blockage. In silico docking of drugs with known structures to a homology model of hERG confirms these observations. The multiple aromatic side chains (eight per hERG channel) are arranged in two concentric rings. These may facilitate multiple drug-specific interactions and may explain the chemical diversity of hERG blockers.

The precise drug docking mechanism depends upon the actual structure of the drug itself and its ability to adopt multiple binding arrangements at different hERG residues within and between subunits (Masetti, M. 2007, J. Comput. Chem. 29, 795-808). On hERG gating the spatial arrangement of these residues may change and it appears that the inactivated state may be preferred by the high-affinity drug blockers.

The Food and Drug Agency in the United States and other Regulatory authorities have mandated that no new drug can be released without as assessment of its hERG affinity and propensity to prolong the QT interval in humans. Such regulation assumes that hERG is a marker for the risk of torsade de pontes and sudden death. However, cardiac depolarization and repolarisation is a complex process generated from multiple competing and complementary ionic currents. High affinity hERG blockers may not prolong QT if they also block other ion channels e.g. verapamil is a potent hERG blocker but does not cause prolonged QT due to a compensatory block of Ca²⁺ channel depolarisation. Therefore other ion channels should be considered as potential risk factors of long QT syndrome.

Current Methods for Assaying the hERG Voltage-gated K⁺ Channel

Identifying pharmaceutical agents that block the hERG channel and thereby pose a risk of potential fatal arrhythmias has become a critical issue for regulatory agencies and the pharmaceutical industry. Sudden death due to torsade de pointes caused by non-cardiovascular drugs such as the antihistamines terfenadine and astemizole led to their withdrawal from the market. Consequently cardiac safety relating to voltage-gated K⁺ channels has become a major concern of regulatory agencies as hERG channel inhibition has been identified as the closest link to QT prolongation. In order to prevent costly attrition it has become a high priority in drug discovery to screen out this inhibitory activity on hERG channels in lead compounds as early as possible. However, a functional accurate, high throughput screening assay for hERG channel activity has proven to be challenging for the industry.

### Patch Clamping

The patch clamp technique allows the study of single or multiple ion channels. The technique can be applied to a wide variety of cells, but is especially useful in the study of excitable cells such as neurons, cardiomyocytes etc. The technique involves an electrode, a glass micropipette that has an open tip (1 μm diameter). The open tip encloses a cell membrane surface or “patch” that generally contains only a few ion channels. In some instances, the micropipette tip is heated to produce a smooth surface that assists in forming a high resistance seal with the cell membrane. The interior of the pipette is filled with a solution matching the ionic composition of the cytoplasm for whole-cell recordings. A silver wire is placed in contact with this solution and conducts electrical current to the amplifier. The investigator can add drugs to this solution to study their effect on specific ion channels.

The micropipette is pressed against the cell membrane and suction is applied to assist in the formation of a high resistance seal (or gigaseal) between the glass and the cell membrane. The high resistance makes it possible to electronically isolate the currents measured across the membrane patch with little competing noise.

Several variations of the technique exist: the inside-out and outside-out techniques are called “excised patch” techniques, because the patch is excised (or removed) from the cell. Cell-attached and excised patch techniques are used to study the behavior of individual ion channels in the section of membrane attached to the electrode. Whole-cell patch and perforated patch clamping allow the electrical behavior of the entire cell to be studied.

Cell-attached or on-cell patch: the electrode is sealed to the membrane patch and the cell remains intact, allowing for the recording of currents through single ion channels without disrupting the cell. Voltage-gated ion channels such as hERG can be clamped at different membrane potentials using the same on cell patch.

Inside-out patch: after the gigaseal is formed, the micropipette is quickly withdrawn, thus ripping a portion of membrane off the cell. The resultant patch remains attached to the micropipette, thus exposing the intracellular surface of the membrane to the external medium. This is useful to manipulate the environment at the intracellular surface of ion channels. For example, channels that are activated by intracellular ligands can then be studied.

Whole-cell recording: these involve recording currents through multiple ion channels at once, over the entire cell. The electrode is attached to the cell, suction is applied causing cell rupture, thus providing access to the intracellular space of the cell. The advantage of whole-cell patch clamp recording over sharp microelectrode recording is that the larger opening at the tip of the patch clamp electrode provides lower resistance and thus better electrical access to the inside of the cell. A disadvantage of this technique is that the volume of the electrode is larger than the cell, so the soluble contents of the cell's interior is slowly replaced by the contents of the electrode (process is known as dialyzing). Thus, any properties of the cell that depend on soluble intracellular contents will be altered. Therefore Whole-cell recording, must be performed quickly.

Outside-out patch: after the whole-cell patch is formed, the electrode is slowly withdrawn allowing the membrane to bleb from the cell. When the electrode is pulled far enough, this bleb will detach from the cell and reform as a membrane on the end of the electrode, with the original outside of the membrane facing outward from the electrode. Single channel recordings are possible if the membrane bleb is small enough. Outside-out patching gives the opportunity to examine the properties of an ion channel when it is isolated from the cell, and exposed to different solutions on the extracellular surface of the membrane.

Perforated patch: in this variation of whole-cell recording, a gigaseal is generated, but suction is not used to rupture the patch membrane. Instead, the electrode solution contains small amounts of antibiotics, such as amphothericin-B or gramicidin. These form small perforations in the membrane, providing electrical access to the cell interior. This has the advantage of reducing the dialysis effect assocaited withwhole-cell recordings, but also has several disadvantages, i) the access resistance is higher, relative to whole-cell. This will decrease electrical access and increase recording noise. ii) it can take a significant amount of time for the antibiotic to perforate the membrane (10-30 min) and iii) the exposed membrane is weakened and can perforate.

Loose patch: these clamps employ a loose seal rather than the tight gigaseal. A significant advantage is that the pipette can be repeatedly removed from the membrane after recording, and the membrane will remain intact. This allows for repeated measurements in a variety of locations on the same cell without destroying the integrity of the membrane. A major disadvantage is that the contact between the pipette and the membrane is reduced.

Electrophysiological studies using the patch clamp technique in hERG-transfected cells generates the most definitive data on hERG inhibition. However, the assay is extremely time-consuming, costly and technically difficult. See below for a detailed description of the conventional patch clamping procedure.

### Automated Patch Clamping

Currently, the conventional whole-cell patch clamp assay is the most reliable method available to accurately determine the activity of compounds against the hERG voltage-gated K⁺ channel. Unfortunately, the technique is time-consuming and limits the determination of hERG activity to 2 or 3 compounds per electro-physiologist per day.

An automated voltage clamp was described by Dubin, A. E. et al., 2005, (J. Biomol. Screening, 10, 168-181). The PatchXpress 7000A (Axon instruments) involves the use of planar electrode biochips to measure compound activity at the hERG channel. The PatchXpress facilitates a continuous whole cell current recording during compound addition and washout. The device is able to independently record in parallel from each of the 16 wells in the associated SealChip16 electrode array (AVIVA Biosciences). Each well bears a single pore extending from the top extracellular chamber (containing essentially high NaCl and low KCl) to a lower chamber (filled with the high KCl intracellular solution). This set up takes the place of the conventional patch clamp electrode tip.

Briefly, the PatchXpress procedure involves the following. A set of seventy compounds (including 29 moderate hERG blockers) were tested on a HEK293 cell line stably transfected with the hERG ion channel at four point concentrations. A significant difference between conventional and planar electrophysiology involves the manipulation of the cells. In planar devise these are resuspended so that >95% exists as single cells. This is in contrast to the conventional method in which the cells are plated on poly-lysine-coated dishes and allowed to adhere ˜12 h prior to testing.

The intracellular solution was injected into the bottom of each chamber of the SealChipl6 electrode array, and the extracellular solution was administered into the top. During this procedure a positive pressure was maintained from the intracellular side (+10 mm Hg) to keep the hole free of debris. Cells were automatically added (10-30,000 cells) to each well. After which the pressure was switched to −30 mm Hg to attract the suspended cells to each of the 16 hole-electrodes. The “giga-seal” was achieved by maintaining negative pressure. Whole cell access was achieved by rupturing the patch of the membrane over the hole by increasing the negative pressure to −130 mm Hg with a pipette potential of −80 mV. The voltage-depended activation of hERG was obtained using several voltage steps from −60 to +60 mV followed by a −40 mV voltage to elicit deactivating tail currents. The voltage-dependence of activation was also determined after exposure to hERG inhibitors. After initial optimising experiments the cells were challenged with a voltage protocol that activated and deactivated the entire population of hERG channels (+60 mV for 2 s), followed by repolarisation (−40 mV for 6 s) to open the channels and elicit a tail current. The tail currents were monitored for 5 min to ensure rundown during which time the compounds under investigation were added. Currents were monitored continuously during the 9 min exposure to the compound. After washing out of the test compound the hERG blocker astemizole was applied to the cells at its IC₅₀ concentration (11 nM) and at a maximum inhibition concentration (220 nM). This was performed as a quality control criterion for data acceptance.

As a control, the data generated using the automated PatchXpress patch clamping systems were compared to conventional patch clamping. The results indicated that the electrical parameters and voltage dependence of the hERG channel was similar to those generated using conventional whole cell patch clamping. The PatchExpress system identified the activity of the 29 moderately potent hERGblockers. The authors claimed that the automated system should facilitate the acceleration of secondary screening for ion channel modulators and thereby the hERG (and other ion) channel drug discovery process.

The introduction of parallel patch clamping instruments offers the promise of moderate and high fidelity voltage clamp analysis for ion channel drug screening assays. Sorota, S. et al., 2005 (Assay & Drug Dev. Tech. 3, 47-57) describe the evaluation of another automated patch clamping system the IonWorks HT (Molecular Devices). The study compared the IonWorks HT system to conventional patch clamping and an alternative Rb⁺ Efflux Screen to determine if either offered a superior predictive value compared to conventional patch clamping. The IonWorks HT platform is broadly similar to that described for the PatchXpress system however it offers the highest potential for overall throughput in that it is capable of evaluating 384 cells in parallel compared to the 16 for the PatchXpress and Flyscreen 8500 (see below) systems.

In general, using the IonWorks HT platform concentration-effect curves for a panel of known hERG blockers were shifted to higher concentrations compared to conventional voltage clamp studies. The authors concluded that results on known hERG channel blockers generated using the IonWorks HT automated patch clamping system did not outperform those derived from the Rb⁺ Efflux Screen. However in terms of predicting conventional patch clamping measurements neither of these systems fully achieved the acceptance criterion and were considered to be less effective then conventional electrophysiological experiments using whole cell patch clamping.

A further parallel patch clamp device is presently marketed to the end-user, the Flyscreen 8500 (Flyion GmbH, Tubingen, Germany).

### Radiolabelled Binding Assays

A hERG channel radiolabelled binding assay based upon [³H]dofetilide was described by Diaz, G. J. et al., 2004, (J. Pharma. & Tox. Meth., 50, 187-199). To validate the utility of the assay as a screening tool, a series of saturation and competition binding studies were performed. The authors compared the binding affinities of 22 known hERG blockers in the presence of [³H]dofetilide in both intact cells (HEK293 cells stably transfected with hERG) and isolated membranes derived from the same cell line. Five different K⁺ concentrations were investigated. Binding assays were performed at 37° C. using [³H]dofetilide concentrations ranging from 0.16 to 400 nM for 45 min. On completion the assay was terminated and filtered onto a Unifilter 96-well glass filter plates (GF/B) pre-coated with 0.5% polyethyleneimine. The plates were washed, dried and the radioactivity determined using a Packard Topcount scintillation counter after addition of scintillant.

For electrophysiology experiments the cells were resuspended in a bath solution containing a high NaCl (140 mM) and low KCl (5 mM). Borosilicate Patch pipettes was used in combination with the low KCl (20 mM) pipette solution. The current derived from the hERG channel was recorded using either an Axopatch 200A or 700A Multi-clamp Commander along with pClamp data acquisition software. Drug effects were assessed using a stepped voltage clamp protocol ranging from −25, 0 25 and 50 mV for 3 secs followed by a step to −50 mV for 4 sec from a holding potential of −80 mV clamp pulses were applied every 15 sec. A total of 56 structurally diverse drugs at 5 and 60 mM K⁺ were assayed using this method and the Ki values generated were compared to functional IC₅₀ values of hERG current block obtained using whole-cell patch clamp.

The authors claimed i) that a good correlation existed between the data generated using the radiolabelled binding assays and the whole cell patch clamp method and ii) the simplicity, predictability and adaptability to high-throughput platforms makes the [³H]dofetilide membrane binding assay a useful tool for screening and ranking compounds for their potential to block the hERG K⁺ channel.

A similar hERG radiolabelled binding assay was developed and validated by Chiu, P. J. S. et al., 2004, (J. Pharmacol. Sci., 95, 311-319) using the potent hERG channel blocker [³H]Astemizole and the HEK293-hERG cell line. The assay was validated with 32 known hERG channel blockers with diverse structures and the binding assay results were compared to electrophysiological studies. Once again the conclusion was that the [³H]Astemizole radiolabelled binding assay was extremely rapid, low cost and capable of detecting hERG inhibitors. However, as with all radioactive assays the safe disposal of contaminated waste remains a significant issue.

### Membrane-Potential-Sensitive Florescent Dyes

A cell-based fluorescence assay using membrane-potential-sensitive florescent dyes and a CHO cell line stably-transfected with hERG was described by Dorn, A. et al., 2005, (J. Biomolecular Screening, 10, 339-347). The assay allows the semi-automated screening of compounds for hERG activity in 384-well plates and is sufficiently rapid for treating a large number of compounds (10,000 data points per day). The florescent-based assay is relatively robust as determined by the Z factor >0.6. The authors claim that the data generated were in “qualitative” agreement with those from patch-clamp electrophysiological analysis however, “quantitative” differences did exist between florescence and electrophysiological methods.

The assay exploits the properties of dyes that upon alteration of the cell membrane potential relocate from the outside to the inside of cells (or vice versa) thus causing an alteration in the florescence intensity. The distribution of the florescent compound bis-oxonol-DiBAC₄ across a membrane depends upon the electrical potential of the membrane. Depolarised cells accumulate the negatively charge oxonol dye and exhibit increased fluorescence while hyperpolarisation is indicated by a decrease in florescence. Thus if the membrane potential depends on K⁺ conductance the florescence signal change can be used as a marker of K⁺ channel activity.

Although this technique using membrane-potential-sensitive florescent dyes could not match the accuracy of the gold standard in ion channel research (i.e. the patch-clamp technique) the associated lower cost and the higher throughput render them an attractive alternative to electrophysiological tests for screening large numbers of compounds.

Molecular Devices market the fluoro-metric imaging plate reader (FLIPR) membrane Potential Assay Kit (FMP). This has been widely accepted for Na⁺ and K⁺ channel screening. However like other fluorescent dyes the kit measures the change in membrane potential instead of actual channel activity. Baxter, D. F. et al., 2002 (J. Biomolecular Screening 7, 79-85) describe the use of the FLIPR FMP kit on the K⁺ channel hELK-1 while Tang, W., et al., 2001 (J. Biomolecular Screening, 6, 325-331) describe its use on the hERG channel. In both studies, the FLIPR FMP kit was compared to the fluorescent dye bis-oxonol-DiBAC₄ and electrophysiological patch clamping.

The study peformed by Tang W., et al., (2001) involved testing five known hERG blockers (dofetilide, terfenadine, serindole, astemizole and cisapride) against the three functional hERG channel assays systems including a Rb⁺ flux assay. The authors concluded that the bis-oxonol-DiBAC₄ assays was the most economical but exhibited a high false hit rate. This was believed to be due to the interaction of the dye with the test compound. The FLIPR FMP kit generated fewer false hits due to less colour-quenching issues but was considerably more expensive. The non-radioactive Rb⁺ flux assay was considered to be the most effective of all the assays evaluated generating the lowest false hit rate.

### Rb⁺ Efflux Assay

The basis of the Rb⁺ efflux assay is that K⁺ channels can also transport Rb⁺ ions. Since mammalian cells do not have any intrinsic Rb⁺ ions any concentration change can be easily detected by using either atomic absorbance or radioactive Rb⁸⁶.

Rezazadeh, S. et al., (2004) J. Biomolecular Screening, 9, 588-597 described a High-Throughput Rb⁺ Efflux Assay. Briefly, cells were loaded with Rb⁺ using a Rb⁺ containing loading buffer. The cells were then washed with the same solution minus Rb⁺ in order to remove it from the extracellular fluid. Cells were depolarized using an open-channel buffer, which consisted of high KCl (150 mM). To analyze the non-radioactive Rb⁺ concentration of the intracellular fluids, cells were lysed using a 1% Triton X-100 solution.

Approximately 50,000 cells were seeded into a 96-well cell culture plate and allowed to incubate for 24 h at 37° C. in an atmosphere of 95% air supplemented with 5% CO₂. After discarding the medium, open channel buffer supplemented with the drug/hERG blocker (30 nM-300 mM), were added for 2.5 h after which the medium was replaced by a mixture of the Rb⁺ loading buffer supplemented with the same drug. Cells were then washed with a wash buffer plus drug, to remove extracellular Rb⁺. Subsequently, channel opening buffer plus drug were added to the wells to activate the hERG channels. After incubation for 5 min, the supernatant was carefully removed and collected.

Cells were lysed by addition of lysis-buffer and the Rb⁺ content of the cell supernatant and cell lysate was determined using the ICR8000 Ion Channel Reader—Atomic Absorbance Spectrometer.

The authors concluded that the non-radioactive Rb⁺ efflux assay is limited by its low sensitivity for detecting hERG blockers as compared to traditional electrophysiological measurements, but the technique is extremely reliable and efficient for high-throughput hERG screening.

A similar Rb⁺ efflux assay was performed by Tang, W. et al., 2001 (J. Biomolecular Screening, 6, 325-331). The authors evaluated several hERG channel assay methods including Rb⁺ efflux, the fluorescent dye bis-oxonol-DiBAC₄, and FLIPR FMP kit compared to the electrophysiological method of patch clamping. Their conclusion was that the non-radioactive Rb⁺ efflux assay was the most promising of all the assays for a high-throughput approach generating the lowest false-hit rate but lacked the sensitivity associated with the patch clamping format).

### HERG-Lite—An Antibody-Based Chemiluminescent Assay

In order to address the need for an inexpensive, rapid, and comprehensive assay to predict hERG risk early in the drug development process, ChantTest Inc., have developed a novel antibody-based chemiluminescent assay called HERG-Lite (see Wible, B. A. et al., 2005, J. Pharma & Tox Meths. 52, 136-145). HERG-Lite monitors the expression of hERG at the cell surface in two different stable mammalian cell lines. One cell line acts as a biosensor for drugs that inhibit hERG trafficking, while the other predicts hERG blockers.

The HERG-Lite system monitors the expression the surface expression of two different hERG channels. Both engineered cell lines express hERG channels with an HA epitope engineered into the extracellular loop spanning transmembrane domains S1 and S2. The first cell line expresses the wild-type channel, hERG-WT-HA, at high basal levels and is used for identification of drugs that induce trafficking inhibition and decrease surface expression. The second cell line expresses hERG containing a single point mutation (Gly601Ser). This mutation in the extracellular loop between the S5 domain and the pore generates a trafficking deficient channel that is largely retained (˜90%) in the ER. Consequently, the Gly601Ser mutant channels show a reduction in current amplitude. Blockers of the channel acting as pharmacological chaperones convert the miss-folded Gly601Ser channels into their correct conformation and rescue channel expression by allowing export from the ER and thus movement to the cell surface. For hERG blockers, the concentration dependence and magnitude of the rescue of Gly601Ser expression correlates with the potency of the block. Stable expression of each tagged hERG channel in HEK293 cells has generated cell lines that serve as biosensors for compounds with hERG risk.

The study of Wible, B. A. et al., (2005), validated the HERG-Lite assay using a panel of 100 drugs: 50 hERG blockers and 50 non-blockers. The authors claimed that the HERG-Lite system correctly predicted hERG risk for all 100 test compounds with no false positives or negatives. All 50 hERG blockers were detected as drugs with hERG risk and the system categorised all the drugs as either blockers or trafficking inhibitors. Chantest Inc., claim that the HERG-Lite system predicts both channel blockers and trafficking inhibitors in a rapid, cost-effective manner and is a valuable non-clinical assay for drug safety testing.

Predictor hERG Fluorescence Polarisation Assay

Fluorescence polarization is based on the observation of fluorescent molecules in solution, when excited by excitatory polarized light, emit polarized light in a different plane. A molecule's polarization is inversely proportional to the molecule's rotational speed, which is influenced by solution viscosity, absolute temperature, molecular volume and the gas constant.

The Predictor™ hERG kit from Invitrogen is a homogeneous fluorescent assay that uses a simple add-and-read format. The assay is based on the principle of fluorescence polarization where a red fluorescent molecule (Predictor hERG Tracer Red) binds at the hERG channel. This molecule is displaced from the hERG channel by compounds that bind to the channel. The Assay performance of the Predictor assay system is validated using established hERG channel blockers (see http://www.biotek.com/resources/articles/predictor-herg-fluorescence-polarization.html).

The Predictor technology is a homogeneous, fluorescence polarisation-based assay that can be used to identify and characterise the affinity of small molecules for the hERG channel. Piper, D. R. et al., 2008 (Assay & Drug Dev. Tech., 6, 213-223) demonstrated the utility of this system for hERG channel screening by comparing radio-ligand binding assay and patch clamp analysis to the Predictor system. The authors concluded that all three methods generated results that exhibited a good correlation.

Key to the development of this assay was a cell line that expressed high levels of the hERG protein. This was achieved by using a bi-cistronic element that facilitated the coupled expression of both the hERG channel with that of a selectable cell surface marker CD8. A high-expressing clone was isolated by flow cytometry and used to generate membrane preparations that contained >50-fold the typical density of the hERG channel. The combination of a high expressing cell line and the high affinity tracer has enabled an assay to be generated that exhibits a Z-value >0.87 which correlates well with the affinity of test compounds in conventional patch clamp assays.

### Micro-Electrode Arrays

A novel approach for studying the electrophysiological properties of cultured cardiac myocytes is by micro-electrode arrays (MEA). This technology utilizes multi channel recording from an array of embedded and substrate-integrated extra-cellular electrodes. The detected field potentials allow a partial reconstruction of the shape and time course of the underlying action potential. In particular, the duration of action potentials of ventricular myocytes is closely related to the QT interval as determined by an ECG. Whereas the traditional patch clamping type hERG assays limits cardiac repolarisation to just one channel, the MEA format reflects the full range of mechanisms involved in cardiac action potential regulation.

This novel technique was used to study several reference substances including the potent selective HERG K⁺ channels blocker E4031. All these compounds are known to exhibit a prolonged QT effect, and all demonstrated a similar prolongation of the field potential on the MEA (Meyer T. et al., 2004 Drug safety 27 763-772).

Cardiac cells were plated at a high density on the electrode field of a polyethylenimine coated MEA. Recordings were carried out on an MEA 1060 System that allows the simultaneous recording from 60 channels. The ventricular myocytes are able to proliferate and form beating syncytium on the MEAs. The signal generated from the MEA consists of components that reflect the composition of an action potential including the rapid negative components that reflects the influx of Na⁺ ions through voltage-dependent Na⁺ channels, the negative plateau associated with a cardiac action potential and the hERG-mediated repolarisation step. All the signal components generated by the MEA were characterized by the use of respective channel blockers.

The authors concluded that screening compounds in physiological-relevant cells i.e. beating cardiac myocytes for compounds that cause QT prolongation with the MEA technology can overcome the problem associated with using single cell assays of reporting ‘false positives’.

Predictive In Silico Modeling for hERG Channel Blockers

Due to the unique shape of the ligand-binding site and its hydrophobic character, the hERG channel has been shown to interact with pharmaceuticals of widely varying structure often at concentrations similar to the levels of on-target activity. Several in silico approaches have been attempted to predict hERG channel blockage. Some of these computational approaches are designed to filter out potential hERG blockers in the context of virtual compound libraries while others involve understanding the structure-function relationships that governs hERG channel and drug interactions (see Aronov, A. M., 2005, D.D.T., 10 149-155 and That, T. M., et al., 2007 Curr. Med. Chem. 14, 3008-3026).

Commercial hERG Screening

Commercial screening is generally based upon either HEK293 or CHO cells stably transfected with hERG. At present hERG screening is available from a range of companies using several different techniques for example:

Electrophysiology—Cellular Dynamics Inc. using a HEK293-hERG stable cell line.

ChanTest—HEK293- and CHO-hERG cells. ChanTest also offer a fluorescent-based screen for monitoring hERG expression and trafficking at the cell surface.

Automated patch clamping—Patch express 700A screening platform is available from Axon Instruments. This company uses a CHO-hERG stable cell line.

Flourescent Polarization Assay—Invitrogen offer the Select Screen hERG screening service based on FP technology.

### Enzyme Complementation

The β-galactosidase derived from Escherichia coli is a tetrameric enzyme with a MW of 464,000 and each identical subunit contains 1021 amino acids. The enzyme is encoded by the lacZ gene located within the lac operon. In E. coli, β-galactosidase complementation is possible. This involves the expression β-galactosidase fragments (α and ω, amino and carboxyl-terminal domains respectively) that by themselves are enzymatically inactive but, when expressed together interact or complement generating a functional β-galactosidase activity.

Ullmann et al., 1965, (J. Mol. Biol., 12, 918-923) described the complementation of β-galactosidase in E. coli. A peptide was found (Peptide ω) that was present in extracts of various mutants (ω donors) of the lacZ gene. The ω-peptide complemented β-galactosidase activity when added to extracts containing a β-galactosidase negative mutant (ω-acceptors). The w enzyme acceptor peptide (EA) has since been found to lack residues 11-41, and is frequently referred to as the M15 protein, since it is a product of the lacZ M15 allele. Sucrose density assessments suggested a MW of 30,000 to 40,000 for the ω-peptide.

A following publication by Ullmann et al. 1967 (J. Mol. Biol., 24, 339-343) described how protein extracts from various β-galactosidase-negative mutants were screened for their capacity to complement with extracts of partial deletions of the operator-proximal segment (α) of the LacZ gene.

Zamenhof, P. and Villarejo, M. 1972, (J. Bacteriol., 110, 171-178) demonstrated 6-galactosidase complementation in vivo using a library of 16 lacZ genes that had been prematurely terminated. Functional activity was generated in a β-galactosidase deficient mutant strain upon introduction of specific gene fragments corresponding to a peptide that contained a small deletion in the N-terminal region of the enzyme monomer.

Since then, many sequence variants of donor and acceptor species of β-galactosidase have been described, reviewed by Eglen, R. 2002, Assay and Drug Development Technologies, 5, 97-105; DiscoveRx). In particular, a variation developed by DiscoveRx is a system for complementation of a small 4 kDa a fragment donor (ED) peptide (termed “ProLabel”) with a ω-deletion mutant of the enzyme acceptor (EA). Further work reviewed by Olson, K. & Eglen, R., 2007, Assay and Drug Development Technologies, 5, 137-144) describes a 47-mer enzyme donor (ED) sequence.

In addition to β-galactosidase enzyme complementation, complementation is a phenomenon now reported for other proteins, including dihydrofolate reductase (Remy, I. & Michnick, S., 2001, Proc. Natl. Acad. Sci. USA., 98, 7678-7683), β-lactamase (Wehrman, T. et al., 2002, Proc. Natl. Acad. Sci. USA, 99, 3469-3474), luciferase (Ozawa T., et al., 2001, Anal. Chem., 73, 2516-2521), ubiquitinase (Rojo-Niersbach E et al., 2000, Biochem. J., 348, 585-590), alkaline phosphatase (Garen, A. & Garen, S., 1963, J. Mol. Biol. 7, 13-22) and tryptophan synthase (Yanofsky, C. & Crawford, I. P., 1972, Enzymes, 7, 1-31).

Henderson et al., (1986 Clinical Chemistry, 32, 1637-1641: Microgenics) describe the genetic engineering of β-galactosidase, leading to the development of a homogeneous immunoassay system.

U.S. Pat. No. 5,120,653 (Microgenics) describes a vector comprising a DNA sequence coding for an enzyme-donor polypeptide.

U.S. Pat. No. 5,643,734 and U.S. Pat. No. 5,604,091 (Microgenics) describe methods and compositions for enzyme complementation assays for qualitative and quantitative measurement of an analyte in a sample.

PCT WO 2003/021265 (DiscoveRx) describes a genetic construct designed for an intracellular monitoring system consisting of biologically active fusion proteins comprising a sequence encoding an enzyme donor (ED) sequence fused in frame to a sequence encoding a mammalian protein of interest, where the fusion protein has the function of the natural protein. Furthermore, a vector is described comprising a transcriptional and translational regulatory region functional in a mammalian cell, a sequence encoding the ED joined to a multiple cloning site, an enzyme acceptor (EA) protein or enzyme acceptor sequence encoding such protein that is complemented by the ED to form a functional enzyme such as β-galactosidase. Mammalian cells are employed that are modified to provide specific functions.

U.S. Pat. No. 7,135,325 (DiscoveRx) describes short enzyme donor fragments of β-galactosidase of not more than 40 amino acids.

PCT WO 2006/004936 (DiscoveRx) describes methods for determining the intracellular state of a protein as well as modifications to the protein. The method involves introducing a surrogate fusion protein comprising a member of an enzyme fragment complementation complex and a target protein. After exposing cells transformed with the surrogate fusion protein to a change in environment (e.g. a candidate drug), the cells are lysed, the lysate separated into fractions or bands by gel electrophoresis and the proteins transferred by Western blot to a membrane. The bands on the membrane are developed using the other member of the enzyme fragment complementation complex and a substrate providing a detectable signal.

US2007/0105160 (DiscoveRx) describes methods and compositions for determining intracellular translocation of proteins employing β-galactosidase fragments that independently complex to form an active enzyme. Engineered cells have two fusion constructs: one fragment bound to a protein of interest; and the other fragment bound to a compartment localizing signal. The cells are used to screen compounds for their effect on translocation.

### Technical Problem

There is a need within the toxicological and pharmaceutical industries to comply with regulatory requirements, mandated by the US Food and Drug Agency, for methods which assess the hERG affinity and propensity to prolong the QT interval in humans of any new drug or medicament. In particular, there is a need for accurate and reliable methods which are not labour intensive but are amenable to high throughput screening.

The present invention addresses these needs and problems and provides novel methods, proteins, nucleotide sequences, vectors, transfected or transformed cells, uses and kits as summarized below.

### SUMMARY OF THE INVENTION

According to a first aspect of the present invention, there is provided a method of testing for the binding of a ligand to a human Ether-a-go-go-related (hERG) voltage-gated potassium ion channel protein in an enzyme complementation assay, the method comprising:
a) providing a fluid sample comprising an hERG voltage-gated potassium ion channel protein comprising an N and a C terminus wherein one terminus is fused to an enzyme fragment which acts as an enzyme donor and the other terminus is fused to an enzyme fragment which acts as an enzyme acceptor;
b) adding a ligand to said fluid sample to allow binding of the ligand to the hERG voltage-gated potassium ion channel protein to alter the distance between the termini and thereby effect enzyme complementation between the enzyme donor and the enzyme acceptor to generate an active enzyme;
c) adding a substrate of the active enzyme to the fluid sample; and
d) detecting a change in an optical signal resulting from the activity of the active enzyme on the substrate as a measure of ligand binding.

In one aspect, the enzyme fragment is an enzyme acceptor or an enzyme donor selected from the group of enzymes consisting of β-galactosidase, β-lactamase, dihydrofolate reductase, luciferase, ubiquitinase, alkaline phosphatase and tryptophan synthase. Preferably, the enzyme acceptor is a fragment of β-galactosidase and the enzyme donor is a fragment of β-galactosidase. For the avoidance of doubt, the skilled person will understand that the enzyme acceptor fragment and the enzyme donor fragment are different enzyme fragments or peptides.

In another aspect, the enzyme donor is fused to the N terminus and the enzyme acceptor is fused to the C terminus of the hERG voltage-gated potassium ion channel protein.

In a further aspect, ligand binding results in an increase in the optical signal.

In one aspect, the enzyme donor of β-galactosidase has the sequence disclosed in SEQ ID NO: 1 or SEQ ID NO: 2.

In another aspect, the enzyme acceptor of β-galactosidase has the sequence disclosed in SEQ ID NO: 3.

In a further aspect, the method is an homogeneous assay. This has the advantage that it simplifies the workflow and does not require any separation steps

In one aspect, the method is for use in toxicological screening, drug screening or physiological assays. Preferably the method is for toxicological screening.

In a second aspect of the present invention, there is provided a cell-based assay for testing for the binding of a ligand to an hERG voltage-gated potassium ion channel protein in an enzyme complementation assay, the method comprising:
a) providing a cell expressing an hERG voltage-gated potassium ion channel protein comprising an N and a C terminus wherein one terminus is fused to an enzyme fragment which acts as an enzyme donor and the other terminus is fused to an enzyme fragment which acts as an enzyme acceptor;
b) adding a ligand to the cell to allow binding of the ligand to the hERG voltage-gated potassium ion channel protein to alter the distance between the termini and thereby effect enzyme complementation between the enzyme donor and the enzyme acceptor to generate an active enzyme;
c) lysing the cell to provide a cellular lysate;
d) adding a substrate of the active enzyme to the cellular lysate; and
e) detecting a change in an optical signal resulting from the activity of the active enzyme on the substrate as a measure of ligand binding.

In one aspect, the enzyme fragment is an enzyme acceptor or an enzyme donor selected from the group of enzymes consisting of β-galactosidase, β-lactamase, dihydrofolate reductase, luciferase, ubiquitinase, alkaline phosphatase and tryptophan synthase. Preferably, the enzyme acceptor is a fragment of β-galactosidase and the enzyme donor is a fragment of β-galactosidase. For the avoidance of doubt, the skilled person will understand that the enzyme acceptor fragment and the enzyme donor fragment are different enzyme fragments or peptides.

In another aspect, the enzyme donor is fused to the N terminus and the enzyme acceptor is fused to the C terminus of the hERG voltage-gated potassium ion channel protein.

In a further aspect, ligand binding results in an increase in the optical signal.

In one aspect, the enzyme donor of β-galactosidase has the sequence disclosed in SEQ ID NO: 1 or SEQ ID NO: 2.

In another aspect, the enzyme acceptor of β-galactosidase has the sequence disclosed in SEQ ID NO: 3.

In a further aspect, the method is for use in toxicological screening, drug screening or physiological assays. Preferably the method is for toxicological screening.

According to a third aspect of the present invention, there is provided an hERG voltage-gated potassium ion channel protein comprising an N and a C terminus wherein one terminus is fused to an enzyme fragment which acts as an enzyme donor and the other terminus is fused to an enzyme fragment which acts as an enzyme acceptor.

In one aspect, the enzyme fragment is an enzyme acceptor or an enzyme donor selected from the group of enzymes consisting of β-galactosidase, β-lactamase, dihydrofolate reductase, luciferase, ubiquitinase, alkaline phosphatase and tryptophan synthase.

In another aspect, the enzyme acceptor is a fragment of β-galactosidase and the enzyme donor is a fragment of β-galactosidase. Preferably, the enzyme donor is fused to the N terminus and the enzyme acceptor is fused to the C terminus of the hERG voltage-gated potassium ion channel protein.

In a further aspect, the enzyme donor of β-galactosidase has the sequence disclosed in SEQ ID NO: 1 or SEQ ID NO: 2.

In one aspect the enzyme acceptor of β-galactosidase has the sequence disclosed in SEQ ID NO: 3.

In another aspect, the protein has the sequence as disclosed in SEQ ID NO: 5.

According to a fourth aspect of the present invention, there is provided a nucleotide sequence encoding a protein as hereinbefore described.

According to a fifth aspect of the present invention, there is provided a vector comprising a nucleotide sequence as hereinbefore described.

According to a sixth aspect of the present invention, there is provided a host cell transformed with a vector as hereinbefore described.

According to a seventh aspect of the present invention, there is provided a use of a host cell as hereinbefore described for use in toxicological screening, drug screening or physiological assays.

According to an eighth aspect of the present invention, there is provided a kit comprising a vector as hereinbefore described and instructions for its use. Typical instructions, for example, might be how to use the vector to transfect cells or how to use the transfected cells in a method according to the invention as hereinbefore described.

### BRIEF DESCRIPTION OF THE FIGURES

FIG. 1 illustrates a human ventricular action potential.

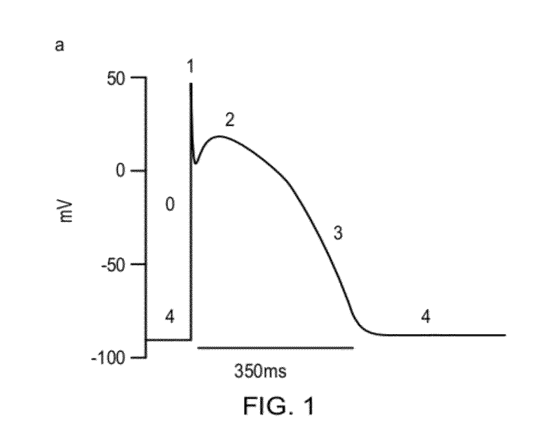

FIG. 2 shows the structure of a single hERG subunit containing six α-helical trans-membrane domains (S1-S6).

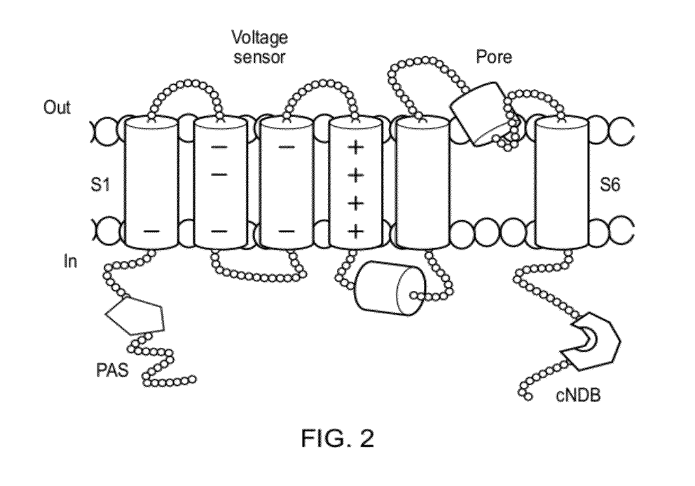

FIG. 3 depicts the amino acid sequence of the β-galactosidase (Enzyme donor)-hERG chimeric protein-β-galactosidase (Enzyme acceptor) chimeric protein.

FIG. 4 is a vector diagram showing the nucleotide sequence of the pCORON1000 (GE Healthcare) mammalian expression vector.

FIG. 5 is a vector diagram showing the nucleotide sequence of the pCORON1000 (GE Healthcare) mammalian expression vector containing the cDNA sequence encoding the β-galactosidase (Enzyme donor)-hERG chimeric protein-β-galactosidase (Enzyme acceptor) chimeric protein.

### DESCRIPTION OF THE SEQUENCE LISTINGS

SEQ ID NO: 1 is the amino acid sequence of the β-galactosidase enzyme donor fragment.

SEQ ID NO: 2 is the amino acid sequence of the β-galactosidase enzyme donor fragment; the 47-mer β-galactosidase enzyme donor being described by Olson and Eglen (Assay and Drug Development Technologies 2007, 5, 97-105).

SEQ ID NO: 3 is amino acid sequence of the β-galactosidase enzyme acceptor fragment.

SEQ ID NO: 4 is the amino acid sequence of the Human ether-a-go-go (hERG, KCNH2 or Kv11.1) voltage-gated potassium ion channel. This amino acid sequence corresponds to that described in Accession number NM 000238.

SEQ ID NO: 5 is the amino acid sequence of the β-galactosidase (Enzyme donor)-hERG chimeric protein-β-galactosidase (Enzyme acceptor) chimeric protein.

SEQ ID NO: 6 is the nucleic acid sequence of the pCORON1000 mammalian expression vector which is available from GE Healthcare.

SEQ ID NO: 7 is the nucleic acid sequence of the pCORON1000 mammalian expression vector (available from GE Healthcare) containing the cDNA sequence encoding the β-galactosidase (Enzyme donor)-hERG chimeric protein-β-galacatosidase (Enzyme acceptor) chimeric protein.

SEQ ID NO: 8 is the amino acid sequence of a linker peptide which is included in SEQ ID NOs: 5. The function of this peptide is to act as a flexible link that connects naturally independent peptides moieties thereby generating a single recombinant chimeric fusion protein. The skilled person will appreciate that other suitable linker peptides could be used to carry out this function.

### DETAILED DESCRIPTION OF THE INVENTION

The present invention provides a cellular hERG assay involving enzyme fragmentation complementation. On hERG channel activation/deactivation with drug or toxic compounds, the distance between the intracellular N and C termini alters. Activation brings the termini closer together. Using recombinant DNA technology it is possible to engineer and generate fusion proteins in which, for example, the donor and acceptor peptides are coupled to the N- and C-terminal of hERG respectively. It will be understood by the skilled person that it is also possible to engineer the alternative combination. While the embodiments described below utilise β-Galactosidase donor and acceptor peptides it will be understood that other embodiments are possible, for example by utilising δ-lactamase, dihydrofolate reductase, luciferase, ubiquitinase, alkaline phosphatase or tryptophan synthase donor or acceptor peptides.

The E. Coli β-Galactosidase N-terminal amino acids e.g. residues 3-92 (or smaller fragments) represent the donor peptide and using recombinant DNA technology, nucleotides encoding these residues can be coupled to the N-terminal cDNA sequence of hERG. The E. coli β-galactosidase acceptor peptide consists of the entire protein minus the residues 11-41. The DNA sequence encoding this modified peptide can then be coupled to the cDNA sequence encoding the C-terminal of the β-Galactosidase donor peptide-hERG fusion protein. (Note—the alternative combination can also be engineered).

The β-galactosidase fragments that would be suitable for use in the present invention could include those described in Applicant's co-pending application “Methods for Testing Binding of a Ligand to a G Protein-Coupled Receptor” (application number GB0822259.8) which is hereby incorporate by reference.

The hERG cytoplasmic N- and C-terminal domains are the sites proposed for linking the 6-galactosidase peptide fragments using recombinant DNA and protein engineering techniques. Indeed, Miranda, et al., 2008 (B.B.A. 1783, 1681-1699) described a library of fluorescent hERG fusion proteins obtained by site-directed coupling of GFP variants into the cytoplasmic N- and C-terminal domains of hERG without loss of biological activity. In addition, US20080286750 (Aviva Biosciences) describes a similar FRET assay linked to a biochip for measurement of ion transport.

The invention is illustrated by reference to the following example.

### Preparation of Genetic Constructs and Transfection of Cells.

The method involves the creation of an artificial cDNA encoding a polypeptide chimera comprising cDNA sequences encoding the human ether-a-go-go related gene (hERG) and two specific fragments of the E. coli β-galactosidase gene. The β-galactosidase peptide fragments are termed the enzyme-acceptor and -donor. The acceptor peptide fragment is capable of enzyme complementation with the β-galactosidase enzyme donor fragment. The enzyme acceptor fragment lacks key amino acids derived from the β-galactosidase peptide. These are complemented by the enzyme donor peptide. When expressed separately the acceptor and donor peptides are enzymatically inactive but, when expressed in combination in the same cellular compartment a functional β-galactosidase activity is generated.

One of the more widely studied examples of a β-galactosidase enzyme acceptor peptide is the X90-acceptor peptide that has a deletion in the last 10 amino acids (1013-1023). The X90 enzyme acceptor peptide exists as a monomer and can be complemented by a corresponding enzyme donor fragment of β-galactosidase, such as CNBr24, a cyanogen bromide digestion product of β-galactosidase consisting of amino acids 990-1023, to reform an enzymatically active tetramer (Welphy et al., 1980, Biochem. Biophys. Res. Common., 93, 223).

The hERG chimera protein is constructed, comprising the β-galactosidase enzyme donor peptide fused to the N-terminal of hERG. This protein moiety is then fused at the C-terminal to the β-galactosidase enzyme acceptor sequence using recombinant DNA techniques. The full length cDNA sequences are available from commercially sources such as Mammalian Gene Collection, NIH, Maryland, USA. The hERG cDNA and protein sequence is described by Accession no. NM_{—}000238.

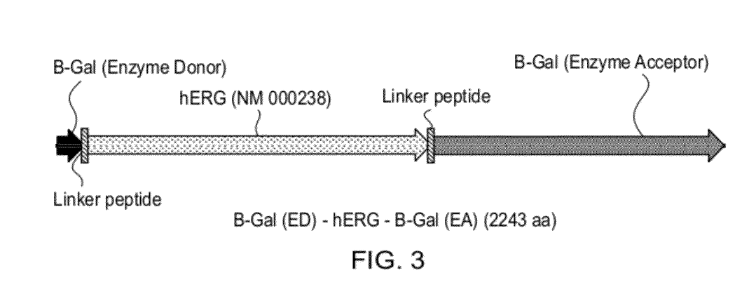

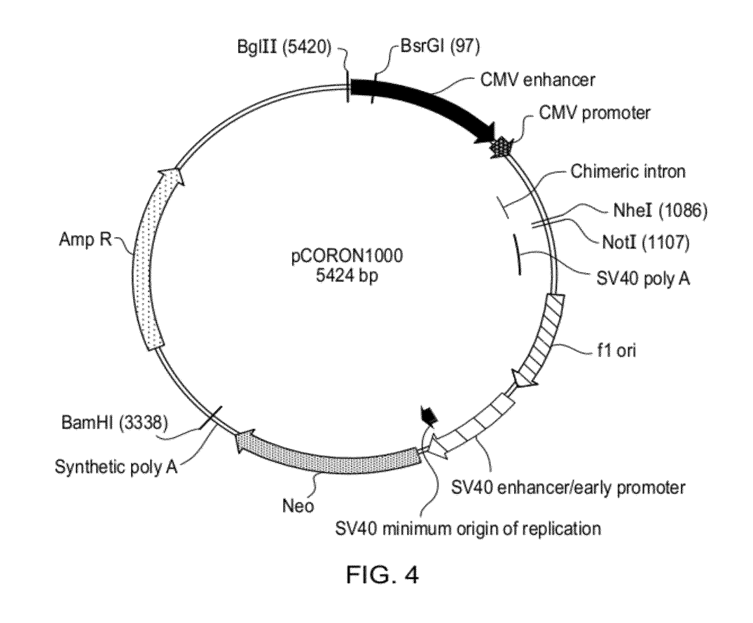

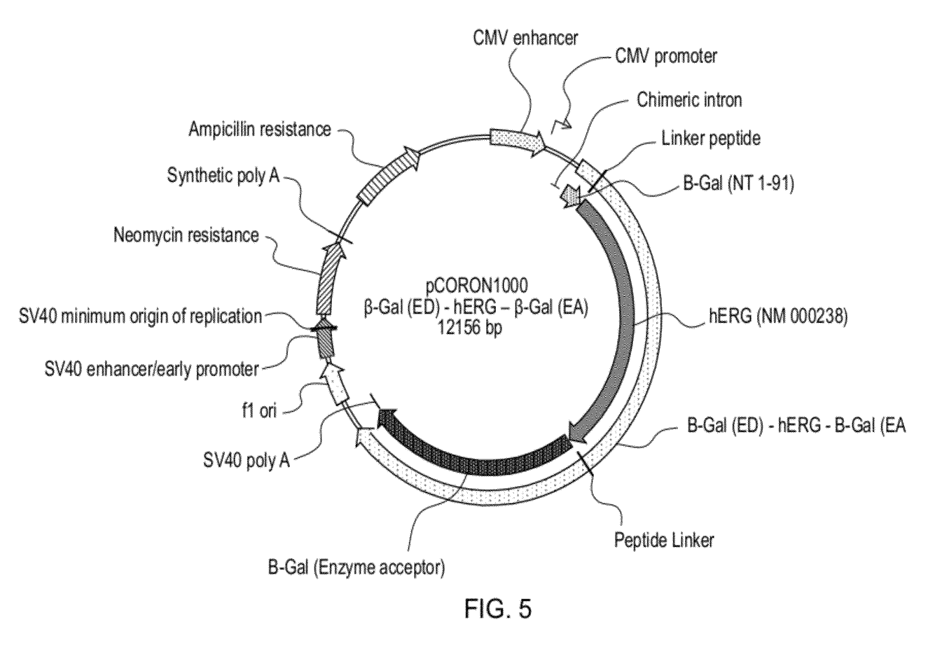

An expression vector (e.g. pCORON1000 from GE Healthcare see FIG. 4 SEQ. ID. No. 6) will be used to generate the β-galactosidase enzyme acceptor-hERG-β-galactosidase enzyme donor chimera using standard molecular biological techniques according to Sambrook and Russell (Molecular Cloning, A Laboratory Manual). The alternative protein chimera can also be generated i.e. β-galactosidase enzyme donor-hERG-β-galactosidaseenzyme acceptor chimera. The amino acid sequence of this chimeric protein is described in FIG. 3 and SEQ. ID. No. 5 and the entire nucleotide sequence of the pCORON1000-β-galactosidase enzyme donor-hERG-β-galactosidase enzyme acceptor chimera is described in FIG. 5 and SEQ. ID. No. 7.

The pCORON1000 Mammalian Expression Vectors carry the human cytomegalovirus immediate-early enhancer/promoter region to promote constitutive expression of a cloned DNA inserts in mammalian cells. The vector also contains the neomycin phosphotransferase gene, a selectable marker for mammalian cells. The pCORON1000 vector can be used for either transient protein expression or for stable expression after the selection (with the antibiotic G-418) of transfected cells that exhibit the appropriate phenotype.

Transfection of target cells (e.g. mammalian cells) using a transfection agent such as Fugene6, with the above-described vector is carried out in accordance with Manufacturer's instructions and following the principles outlined by Sambrook and Russell (Molecular Cloning, A Laboratory Manual, 3^{rd} Edition, Volume 3, Chapter 16, Section 16.1-16.54). For example, Fugene 6 and jetPEI, Roche and Polyplus Transfections respectively. In addition transient viral transduction can also be performed using reagents such as adenoviral vectors (Ng P and Graham F L. Methods Mol Med. 2002; 69, 389-414).

The resulting transfected cells are maintained in culture or frozen for later use according to standard practices. These cells will express the desired hERH-β-galactosidase protein chimera such as β-galactosidase enzyme acceptor-hERG-β-galactosidase enzyme donor chimera protein, as described above.

In one embodiment of the present invention, the β-galactosidase enzyme donor fragment has the amino acid sequence shown in SEQ ID NO: 1.

In another embodiment, the β-galactosidase enzyme donor fragment has the amino acid sequence shown in SEQ ID NO: 2. The 47-mer β-galactosidase enzyme donor being described by Olson and Eglen (Assay and Drug Development Technologies 2007, 5, 97-105).

In another embodiment, the β-galactosidase enzyme acceptor fragment has the amino acid sequence shown in SEQ ID NO: 3.

In another embodiment, the Human ether-a-go-go (hERG, KCNH2 or Kv11.1) has the amino acid sequence shown in SEQ ID NO: 4. This amino acid sequence corresponds to that described in Accession number NM_{—}000238.

### Assay Method

Intact cells expressing the β-galactosidase enzyme acceptor-hERG-β-galactosidase enzyme donor chimeric protein are allowed to come into contact in a tube (microwell) in the presence of a suitable buffer. In the presence of a suitable hERG channel activator such as:
NS1642 Casis, O. et al., (2006), Mol. Pharmacol. 69, 658-665.
PD307243 Xu, X. et al., (2008), Mol. Pharmacol. DOI 10.1124/mol 108.045591.
RPR260243 Kang, T. et al., (2005), Mol. Pharmacol. 67, 827-836.
Mallotoxin Zeng. et al., (2006), J. Pharmacol. Exp. Ther. 319, 957-962.
NS3623 Hansen, et al., (2006), Mol. Pharmacol. 70, 1319-1329.
PD118057 Zhou, et al., (2005), Mol. Pharmacol. 68, 876-884.

The hERG channel becomes activated, leading a change in the proximity of the intracellular N- and C-terminal domains. Activation brings the termini closer together. Therefore on hERG activation the attached β-galactosidase enzyme acceptor and enzyme donor peptides are brought closer together, leading to β-galactosidase enzyme complementation and hence functionl enzyme activity. Upon lysis of the cells, with a suitable lysis agent (e.g. detergent, Triton X100 or Tween20) and the addition of a suitable β-galactosidase substrate such as the pro-luminescent 1,2-dioxetane substrate (alternative substrates include, for example, 5-acetylaminofluorescein di-b-D-galactopyranoside (X-gal) from Invitrogen; 5-Iodo-3-indolyl-beta-D-galactopyranoside from Sigma; or 5-acetylaminofluorescein di-b-D-galactopyranoside from Invitrogen), an optical signal is generated which can be detected by, for example, a photomultiplier device.

In this system, a signal increase arises from a higher degree of β-galactosidase complementation which is directly proportional to the potency of hERG channel activator.

It will be understood that this method can be adapted to use recombinant proteins in an a cellular approach using a cell-free system utilising cell membranes. The use of cell-permeable β-galactosidase substrates will facilitate the generation of a live cell-based assay.

Screening Assay Method for hERG

Cells which express the appropriate β-galactosidase enzyme acceptor-hERG-β-galactosidase enzyme donor chimeric protein (described above) are transferred into a 96-well (20,000 pre well) or 384 (5,000 cells per well) culture plate and incubated overnight at 37° C. in a 5% atmosphere of CO₂. An aliquot (e.g. 5 μl) of a suitable test compound or hERG activator (e.g. PD118057, PD307243 etc) or hERG blocker (e.g. pimozide, astemizol, dofetilide, flumarizine, cisapride, oxatomide, mibefradil, ketoconazole or terfenadine) dissolved or suspended in a non-toxic solvent is added to each well and the plate incubated for 1 hour at 37° C. in a 5% atmosphere of CO₂ to allow enzyme complementation to occur. A lysis reagent (such as an appropriate detergent, e.g. Triton X-100 or Tween 20) is added to each well and the plate incubated for 5 minutes. An appropriate luminescent substrate of β-galactosidase (e.g. 5-acetylaminofluorescein di-b-D-galactopyranoside (X-gal) from Invitrogen; 5-Iodo-3-indolyl-beta-D-galactopyranoside from Sigma; or 5-acetylaminofluorescein di-b-D-galactopyranoside from Invitrogen) is added to each well and the plate incubated for 1 to 18 hour (s) at 37° C. in a 5% CO₂ atmosphere. A change in the optical signal (e.g. fluorescence or luminescence) is read using a plate reader or imager (e.g. Leadseeker, GE Healthcare).

While preferred illustrative embodiments of the present invention are described, one skilled in the art will appreciate that the present invention can be practised by other than the described embodiments, which are presented for purposes of illustration only and not by way of limitation. The present invention is limited only by the claims that follow.

1. A method of testing for the binding of a ligand to a human Ether-a-go-go-related (hERG) voltage-gated potassium ion channel protein in an enzyme complementation assay, said method comprising:
a) providing a fluid sample comprising an hERG voltage-gated potassium ion channel protein comprising an N and a C terminus wherein one said terminus is fused to an enzyme fragment which acts as an enzyme donor and the other terminus is fused to an enzyme fragment which acts as an enzyme acceptor;
b) adding a ligand to said fluid sample to allow binding of said ligand to said hERG voltage-gated potassium ion channel protein to alter the distance between the termini and thereby effect enzyme complementation between said enzyme donor and said enzyme acceptor to generate an active enzyme;
c) adding a substrate of said active enzyme to the fluid sample; and
d) detecting a change in an optical signal resulting from the activity of the active enzyme on said substrate as a measure of ligand binding;
wherein said enzyme fragment is an enzyme acceptor or an enzyme donor selected from the group of enzymes consisting of β-galactosidase, β-lactamase, dihydrofolate reductase, luciferase, ubiquitinase, alkaline phosphatase and tryptophan synthase.

2. (canceled)

3. The method of claim 1, wherein the enzyme acceptor is a fragment of β-galactosidase and the enzyme donor is a fragment of β-galactosidase.

4. The method of claim 1, wherein the enzyme donor is fused to the N terminus and the enzyme acceptor is fused to the C terminus of the hERG voltage-gated potassium ion channel protein.

5. (canceled)

6. The method of claim 1, wherein the enzyme donor of β-galactosidase has the sequence disclosed in SEQ ID NO: 1 or SEQ ID NO: 2.

7. The method of claim 1, wherein the enzyme acceptor of β-galactosidase has the sequence disclosed in SEQ ID NO: 3.

8. (canceled)

9. The method of claim 1, for use in toxicological screening, drug screening or physiological assays.

10. A cell-based assay for testing for the binding of a ligand to an hERG voltage-gated potassium ion channel protein in an enzyme complementation assay, said method comprising:
a) providing a cell expressing an hERG voltage-gated potassium ion channel protein comprising an N and a C terminus wherein one said terminus is fused to an enzyme fragment which acts as an enzyme donor and the other terminus is fused to an enzyme fragment which acts as an enzyme acceptor;
b) adding a ligand to the cell to allow binding of said ligand to said hERG voltage-gated potassium ion channel protein to alter the distance between the termini and thereby effect enzyme complementation between the enzyme donor and said enzyme acceptor to generate an active enzyme;
c) lysing the cell to provide a cellular lysate;
d) adding a substrate of said active enzyme to said cellular lysate; and
e) detecting a change in an optical signal resulting from the activity of the active enzyme on said substrate as a measure of ligand binding;
wherein said enzyme fragment is an enzyme acceptor or an enzyme donor selected from the group of enzymes consisting of β-galactosidase, β-lactamase, dihydrofolate reductase, luciferase, ubiquitinase, alkaline phosphatase and tryptophan synthase.

11. (canceled)

12. The method of claim 10, wherein the enzyme acceptor is a fragment of β-galactosidase and the enzyme donor is a fragment of β-galactosidase.

13-14. (canceled)

15. The method of claim 10, wherein the enzyme donor of β-galactosidase has the sequence disclosed in SEQ ID NO: 1 or SEQ ID NO: 2.

16. The method of claim 10, wherein the enzyme acceptor of β-galactosidase has the sequence disclosed in SEQ ID NO: 3.

17. The method of claim 10, for use in toxicological screening, drug screening or physiological assays.

18. An hERG voltage-gated potassium ion channel protein comprising an N and a C terminus wherein one said terminus is fused to an enzyme fragment which acts as an enzyme donor and the other terminus is fused to an enzyme fragment which acts as an enzyme acceptor, wherein said enzyme fragment is an enzyme acceptor or an enzyme donor selected from the group of enzymes consisting of β-galactosidase, β-lactamase, dihydrofolate reductase, luciferase, ubiquitinase, alkaline phosphatase and tryptophan synthase.

19. (canceled)

20. The protein of claim 18, wherein the enzyme acceptor is a fragment of β-galactosidase and the enzyme donor is a fragment of β-galactosidase.

21. (canceled)

22. The protein of claim 18, wherein the enzyme donor of β-galactosidase has the sequence disclosed in SEQ ID NO: 1 or SEQ ID NO: 2.

23. The protein of claim 18, wherein the enzyme acceptor of β-galactosidase has the sequence disclosed in SEQ ID NO: 3.

24. The protein of claim 18 as disclosed in SEQ ID NO: 5.

25. A nucleotide sequence encoding the protein of claim 18.

26. A vector comprising the nucleotide sequence of claim 25.

27. A host cell transformed with a vector according to claim 26.

28. (canceled)

29. A kit comprising the vector of claim 25 and instructions for its use.
